(19) United States
(12) Patent Application Publication
(10) Pub. No.: US 2018/0200326 A1
(43) Pub. Date: Jul. 19, 2018

(54) ANG-(1-7) AND DERIVATIVE OLIGOPEPTIDES FOR THE TREATMENT OF TRAUMATIC BRAIN INJURY AND OTHER COGNITIVE IMPAIRMENTS

(71) Applicant: Arizona Board Of Regents On Behalf Of The University of Arizona, Tucson, AZ (US)

(72) Inventors: Meredith Hay, Tucson, AZ (US); Robin L. Polt, Tucson, AZ (US); Todd Vanderah, Tucson, AZ (US); Tally Milnes, Tucson, AZ (US)

(21) Appl. No.: 15/865,678

(22) Filed: Jan. 9, 2018

Related U.S. Application Data
(60) Provisional application No. 62/444,169, filed on Jan. 9, 2017.

(51) Int. Cl. A61K 38/08; A61P 25/28; A61K 38/14

(52) U.S. Cl. CPC A61K 38/085; A61K 38/14; A61P 25/28

(57) ABSTRACT

The present invention provides oligopeptides, in particular, Ang-(1-7) derivatives, and methods for using and producing the same. In one particular embodiment, oligopeptides of the invention have higher blood-brain barrier penetration and/or in vivo half-life compared to the native Ang-(1-7), thereby allowing oligopeptides of the invention to be used in a wide variety of clinical applications including in treatment of cognitive dysfunction and/or traumatic brain injury.

### CROSS-REFERENCE TO RELATED APPLICATIONS

This application claims the priority benefit of U.S. Provisional Application No. 62/444,169, filed Jan. 9, 2017, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 21, 2018, is named UAPN_003_SL.txt and is 11,716 bytes in size.

### FIELD OF THE INVENTION

The present invention relates to oligopeptides, such as Ang-(1-7) and related derivative oligopeptides, and methods for using the same for the treatment of traumatic brain injury (TBI) and cognitive impairments caused by TBI and other conditions.

### BACKGROUND OF THE INVENTION

Severe traumatic brain injuries (TBI) initiate a cascade of events that lead to a plethora of adverse effects including dramatic elevations of intracranial pressure (ICP) and dysfunction of cerebrovascular regulatory mechanisms essential for survival. Ischemic brain injury is observed universally in those patients who die following severe TBI. Intracranial hypertension (IH) following TBI is associated with direct effects on cerebral perfusion which may be responsible for secondary ischemia. The contributions of both post-traumatic cerebral edema and alteration in cerebral blood volume to ICP appear to vary based on the length of time after the primary mechanical insult. This combination of vasomotor dysfunction and abnormalities in vascular permeability is characteristic of acute inflammation.

The mortality rate from severe TBI in the United States alone is estimated to be about 9-30 deaths per 100,000. Those suffering brain injury requiring medical treatment number 160-300 per 100,000, with approximately 20 percent of patients admitted to treatment facilities sustain a moderate to severe degree of injury as measured by the Glasgow Coma Score (GCS) of 3-12.

There exists a need to provide simplified methodology for treating TBI in mammals, including humans. The present inventions are based on the discovery that native Ang(1-7), related derivative polypeptides, and/or non-peptide agonists that have affinity and agonistic efficacy for the Mas receptor improve a variety of biologic, physiologic, and pathologic parameters. Specifically, it is shown that Mas receptor activation attenuates spatial memory and object recognition impairment caused by congestive heart failure (CHF), pain of various etiologies including cancer-induced bone pain and the neurological sequelae of traumatic brain injury (TBI).

### SUMMARY OF THE INVENTION

Some aspects of the invention provide an oligopeptide that is a non-naturally-occurring angiotensin-(1-7) derivative polypeptide, i.e., “Ang-(1-7) derivative.” Oligopeptides of the invention may have a longer in vivo half-life and/or increased blood-brain barrier penetration than Ang-(1-7). In some embodiments, the oligopeptides of the invention have seven or eight amino acids and have biological activity as an agonist of the Mas receptor.

One particular aspect of the invention provides an oligopeptide derivative of the formula: A¹-A²-A³-A⁴-A⁵-A⁶-A⁷-A⁸ (SEQ ID NO:1), where A¹ is selected from the group consisting of aspartic acid, glutamic acid, alanine, and glycosylated forms thereof; A² is selected from the group consisting of arginine, histidine, lysine, and glycosylated forms thereof; A³ is selected from the group consisting of valine, alanine, isoleucine, leucine, and glycosylated forms thereof; A⁴ is selected from the group consisting of tyrosine, phenylalanine, tryptophan, and glycosylated forms thereof; A⁵ is selected from the group consisting of isoleucine, valine, alanine, leucine, and glycosylated forms thereof; A⁶ is selected from the group consisting of histidine, arginine, lysine, and glycosylated forms thereof; A⁷ is selected from the group consisting of proline, glycine, serine, and glycosylated forms thereof; and A⁸ can be present or absent, wherein when A⁸ is present, A⁸ is selected from the group consisting of serine, threonine, hydroxyproline, and glycosylated forms thereof, provided (i) at least one of A¹-A⁸ is optionally substituted with a mono- or di-carbohydrate; or (ii) when A⁸ is absent: (a) at least one of A¹-A⁷ is substituted with a mono- or di-carbohydrate, (b) A⁷ is terminated with an amino group, or (c) a combination thereof.

In some embodiments, carbohydrate comprises glucose, galactose, xylose, fucose, rhamnose, lactose, cellobiose, melibiose, or a combination thereof. In other embodiments, A⁸ is serine or a glycosylated form thereof, or A⁸ is absent and A⁷ is serine or a glycosylated form thereof. In some embodiments, only the C-terminal amino acid is glycosylated (e.g., A⁸ or A⁷ when A⁸ is absent).

Still in other embodiments, (i) A⁸ is terminated with an amino group; or (ii) when A⁸ is absent, A⁷ is terminated with an amino group. Within these embodiments, in some instances (i) A⁸ is serine that is optionally glycosylated (e.g., with glucose or lactose); or (ii) when A⁸ is absent, A⁷ is serine that is optionally glycosylated (e.g., with glucose or lactose). Still in other instances, when A⁸ is absent and A⁷ serine that is glycosylated with glucose. Within the latter instances, in some cases A⁷ is terminated with an amino group. In some embodiments, whether or not the Ang(1-7) derivative is terminated with an amino group, the C-terminal amino acid (A⁸ or A⁷ when A⁸ is absent) is the only glycosylated amino acid.

Yet in other embodiments, A¹ is aspartic acid; A² is arginine; A³ is valine; A⁴ is tyrosine; A⁵ is isoleucine; A⁶ is histidine; and (i) A⁸ is absent and A⁷ is terminated with an amino group or A⁷ is a glycosylated serine, or (ii) A⁸ is serine terminated with an amino group. Within these embodiments, in some cases A⁸ is a glycosylated serine. Still in other cases, A⁸ is absent and A⁷ is a glycosylated serine that is terminated with an amino group.

Another aspect of the invention provides a glycosylated Ang-(1-7) derivative having eight amino acids or less, typically seven or eight amino acids (e.g., amino acid residues). In some embodiments, the glycosylated Ang-(1-7) derivative is glycosylated with xylose, fucose, rhamnose, glucose, lactose, cellobiose, melibiose, or a combination thereof. Still in other embodiments, the carboxylic acid end of said glycosylated Ang-(1-7) derivative is substituted with an amino group.

Other aspects of the invention provide methods for treating traumatic brain injury and/or a cognitive dysfunction and/or impairment in a subject (i.e., a subject diagnosed as having or suspected to have the stated condition or injury) by administering a therapeutically effective amount of an oligonucleotide of the invention. In general, oligopeptides of the invention can be used to treat any clinical condition that can be treated with Ang-(1-7).

In some embodiments, the oligopeptides of the invention may be used to reduce or eliminate one or more symptoms of traumatic brain injury (e.g., concussion and penetrating brain injury) including neurodegeneration, neuronal loss, and/or cognitive impairment. In other embodiments, the oligopeptides of the invention may be used to reduce or eliminate cognitive impairment, neurodegeneration, and/or neuronal loss caused by or associated with vascular contributions to cognitive impairment and dementia (“VCID”) including, for example, reduced attention, memory loss, psychomotor slowing, and diminished executive function. Specific conditions that are associated with cognitive impairment and/or VCID, and that are amenable to treatment using the inventive oligopeptides include, for example, cognitive impairment caused by or associated with congestive heart failure, cardiovascular disease, hypertension, stroke, postoperative cognitive defects and/or delerium, dementia including age-related dementia, vascular dementia, and Alzheimer's disease. In other embodiments, the oligopeptides of the invention may be used to reduce or eliminate one or more symptoms of HIV-induced neuropathy, diabetic neuropathy, and chemotherapeutic neuropathy, including neurodegeneration, neuronal loss, and/or cognitive impairment.

In some embodiments, the inventive oligopeptides are administered at a dosage of about 0.1-50 mg/kg, including for example at least about 0.25, 0.50, 0.75, 1.0, 1.5, 2.0, 2.5, 3.0, 4.0, 5.0, 10, 15, 20, 25, 30, or 40 mg/kg. The oligopeptides may be administered QD, bid, tid, qid, or more as necessary to obtain the desired clinical outcome. The oligopeptides may be administered orally or by injection (intravenous, subcutaneous, intramuscular, intraperitoneal, intracerebroventricular, or intrathecal), or by inhalation.

### BRIEF DESCRIPTION OF THE DRAWINGS

FIG. 1 is a graph showing some of the oligopeptides of the invention and native Ang-(1-7) to activate human umbilical vascular endothelial cells (HUVEC) in culture.

FIG. 2 is a graph showing NO production assay results for native Ang-(1-7) and oligopeptides PN-A3, PN-A4 and PN-A5 of the invention.

FIG. 3A is a graph showing the select Mas receptor antagonists A779 blocks NO production induced by oligopeptide PN-A5 of the invention.

FIG. 3B is a graph showing the averaged effect of the select Mas receptor antagonists A779 on NO production induced by oligopeptide PN-A5.

FIG. 4 is a graph showing the effects of oligopeptide PN-A5 on heart failure induced object recognition memory impairment.

FIG. 5 is a graph showing the effects of oligopeptide PN-A5 on heart failure induced spatial memory impairment.

FIG. 6 is a line graph showing the discrimination ratio of experimental animals in a novel object recognition test after an acute traumatic brain injury (TBI).

FIG. 7A is model of the three-dimensional structure of native Ang(1-7). FIG. 7B is a computational model of various glycosylated Ang(1-7) derivatives.

FIG. 8 is a line graph showing the in vitro serum half-life of native Ang(1-7) and various derivatives.

FIGS. 9A and 9B are a series of line graphs showing the serum (A) and CSF (B) concentration of native Ang(1-7) and PN-A5.

### DETAILED DESCRIPTION OF THE INVENTION

### Definitions

The term “native Ang-(1-7)” refers to the naturally-occurring Ang(1-7) polypeptide having the amino acid sequence Asp-Arg-Val-Tyr-Ile-His-Pro (SEQ ID NO: 2).

The term “Ang-(1-7) derivative” refers to oligopeptide in which one or more amino acid residue is either modified or different than the amino acid residue of the corresponding native Ang-(1-7). The term “Ang-(1-7) derivative” also includes oligopeptide of eight amino acid residues as discussed in more detail below.

By “PN-A2” is meant the Ang(1-7) derivative of SEQ ID NO: 3, which is has the amino acid sequence of native Ang(1-7) except that Pro⁷ comprises a C-terminal amidation (NH₂).

By “PN-A3” is meant the Ang(1-7) derivative of SEQ ID NO: 9, which is has the amino acid sequence of native Ang(1-7) with the addition of a serine at the C-terminus (i.e., Ser⁸) and wherein Set′ is glucosylated and comprises a C-terminal amidation (NH₂).

By “PN-A4” is meant the Ang(1-7) derivative of SEQ ID NO: 9, which is has the amino acid sequence of native Ang(1-7) with the addition of a serine at the C-terminus (i.e., Ser⁸) and wherein Ser⁸ is lactosylated and comprises a C-terminal amidation (NH₂).

By “PN-A5” is meant the Ang(1-7) derivative of SEQ ID NO: 13, which is has the amino acid sequence of native Ang(1-7) except that Pro⁷ is substituted by Ser⁷ and wherein Ser⁷ is glucosylated and comprises a C-terminal amidation (NH₂).

By “PN-A6” is meant the Ang(1-7) derivative of SEQ ID NO: 13, which is has the amino acid sequence of native Ang(1-7) except that Pro′ is substituted by Ser⁷ and wherein Ser⁷ is lactosylated and comprises a C-terminal amidation (NH₂).

The term “carbohydrate” refers to pentose and hexose of empirical formula (CH₂O)ₙ, where n is 5 for pentose and 6 for hexose. A carbohydrate can be monosaccharide, disaccharide, oligosaccharide (e.g., 3-20, typically 3-10, and often 3-5 monomeric saccharides are linked together), or polysaccharide (e.g., greater than 20 monomeric saccharide units). More often, the term carbohydrate refers to monosaccharide and/or disaccharide. However, it should be appreciated that the scope of the invention is not limited to mono- or di-saccharides. Often the terms “carbohydrate” and “saccharide” are used interchangeably herein.

The term “oligopeptide” as used throughout the specification and claims is to be understood to include amino acid chain of any length, but typically amino acid chain of about fifteen or less, often ten or less, still more often eight or less, and most often seven or eight.

It should be appreciated that one or more of the amino acids of Ang-(1-7) can be replaced with an “equivalent amino acid”, for example, L (leucine) can be replaced with isoleucine or other hydrophobic side-chain amino acid such as alanine, valine, methionine, etc., and amino acids with polar uncharged side chain can be replaced with other polar uncharged side chain amino acids. While Ang-(1-7) comprises 7 amino acids, in some embodiments the oligopeptide of the invention has eight or less amino acids.

By “glycosylated,” is meant the covalent attachment to that amino acid of a mono-, di-, or polysaccharide. The glycosylation may be N-linked or O-linked, as appropriate. For example, N-linked glycosylation may occur at the R-group nitrogen in asparagine or arginine, and 0-linked glycosylation may occur through the R-group hydroxyl of serine, threonine, and tyrosine. Suitable carbohydrates include, for example, monosaccharides such as glucose, galactose, fructose, xylose, ribose, arabinose, lyxose, allose, altrose, mannose, fucose, and rhamnose, disaccharides such as sucrose, lactose, maltose, trehalose, melibiose, cellobiose, higher-order structures such as sorbitol, mannitol, maltodextrins, and farinose, and amino sugars such as galactosamine and glucosamine. In some particular embodiments, the polypeptide is glycosylated with glucose, lactose, cellobiose, melibiose, β-D-glucose, β-D-lactose, β-D-cellobiose, or β-D-melibiose.

The term “combinations thereof,” which reference to any modifications (e.g, carbohydrate modifications) of Ang-(1-7) derivatives refers to oligopeptides in which two, three, four, five, six, seven, or eight of the individual amino acids are modified by the attachment of a carbohydrate. For Ang-(1-7) derivatives having a plurality of carbohydrate modifications, the modifying carbohydrates may be the same on every modified amino acid, or the several modified amino acids may comprise a mixture of different carbohydrates.

“A therapeutically effective amount” means the amount of a compound that, when administered to a mammal, at an appropriate interval and for a sufficient duration for treating a disease, is sufficient to effect such treatment for the disease. The “therapeutically effective amount” will vary depending on the compound, the disease and its severity, physiological factors unique to the individual including, but not limited to the age, weight, and body mass index, the unitary dosage, cumulative dosage, frequency, duration, and route of administration selected.

“Prevent,” when used in connection with the occurrence of a disease, disorder, and/or condition, refers to reducing the risk of developing the disease, disorder and/or condition for which the subject is at risk of developing

“Treat” refers to any method used to partially or completely alleviate, ameliorate, relieve, inhibit, prevent, reduce severity of one or more symptoms or features of a particular disease, disorder, and/or condition in a subject diagnosed as having that disease or disorder.

The terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “subject” or “patient” refers to any organism to which a composition of this invention may be administered, e.g., for experimental, diagnostic, and/or therapeutic purposes. Typical subjects include animals (e.g., mammals such as mice, rats, rabbits, dogs, cats, non-human primates, and humans).

By “dosing regimen” is meant a set of unit doses (e.g., one, two, three, four, or more) that is/are administered individually to a subject, typically separated by periods of time. In some embodiments, a dosing regimen comprises one or a plurality of doses each of which are separated from one another by a time period. The time period separating individual doses may have a fixed or variable duration, or the therapeutic agent may be administered on an as-need basis. A dosing regimen may span one day, multiple days, multiple weeks, multiple months, or be administered for the lifetime of the subject (e.g., 1, 2, 3, 4, 5, 6, 7, 10, 14, 21, or 28 days, or 1, 2, 3, 4, 5, 6, 9, or 12 months or more). In some embodiments, the therapeutic agent is administered once a day (QD), twice a day (BID), three times a day (TID), four times a day (QID), or less frequently (i.e., every second or third day, one each week, or once each month).

Unless defined otherwise, all technical and scientific terms used herein have the meaning commonly understood by a person skilled in the art to which this invention belongs.

Oligopeptides of the Invention:

The renin-angiotensin system (RAS), well known for roles in blood pressure regulation and fluid homeostasis, was recently implicated in metastatic bone disease including inflammation, angiogenesis, tumor cell proliferation, and migration. Angiotensin II (Ang II) is the major end product of the RAS through cleavage by Angiotensin Converting Enzyme (ACE). This nonapeptide binds to and activates two G-protein coupled receptors (GPCRs): angiotensin II receptor type 1 (AT1) and type 2 (AT2). Physiological effects such as vasoconstriction, inflammation, fibrosis, cellular growth/migration, and fluid retention are reported for AT1 and AT2. Ang II is cleaved by ACE2 to yield Angiotensin-(1-7) (Ang-(1-7)), a biologically active heptapeptide. In contrast to Ang II, Ang-(1-7) binds to the GPCR, Mas receptor (MasR; Kd=0.83 nM) with 60-100 fold greater selectivity over the AT1 and AT2 receptors. Activation of the MasR elicits effects opposite to those of the Ang II/AT1/AT2 axis including having anti-inflammatory and antidepressant activities.

Some aspects of the invention provide oligopeptides that are derivatives of Ang-(1-7). As discussed above, the term “derivative” of Ang-(1-7) refers to an oligopeptide whose amino acid sequence of any one or more of Ang-(1-7) is modified (e.g., via methylation, presence of a functional group, such as hydroxy group on proline), attached to a carbohydrate, is replaced with corresponding D-amino acid or an “equivalent amino acid” as defined above, and/or the terminal amino group end or the carboxyl end of Ang-(1-7) is modified, for example, the carboxylic acid end can be modified to be an amide, an amine, a thiol, or an alcohol functional group, or one in which an additional amino acid residue is present compared to native Ang-(1-7). It should be appreciated that the term “Ang-(1-7) derivative” excludes the native Ang-(1-7), i.e., amino acid sequences of endogenous Ang-(1-7) without any modification.

In some embodiments, oligopeptides of the invention have the amino group on the carboxylic acid terminal end (i.e., the —OH group of the carboxylic acid is replaced with —NR^{a}R^{b}, where each of R^{a} and R_{b} is independently hydrogen or C₁-C₆ alkyl) and/or have one or more amino acid residues that are (i) replaced with a corresponding D-amino acid, (ii) glycosylated, (iii) replaced with another amino acid, (iv) or a combination thereof.

In one particular embodiment, the oligopeptide of the invention is Ang-(1-7) derivative of the formula: A¹-A²-A³-A⁴-A⁵-A⁶-A⁷-A⁸ (SEQ ID NO:1), where A¹ is selected from the group consisting of aspartic acid, glutamic acid, alanine, and a derivative thereof; A² is selected from the group consisting of arginine, histidine, lysine, and a derivative thereof; A³ is selected from the group consisting of valine, alanine, isoleucine, leucine, and a derivative thereof; A⁴ is selected from the group consisting of tyrosine, phenylalanine, tryptophan, and a derivative thereof; A⁵ is selected from the group consisting of isoleucine, valine, alanine, leucine, and a derivative thereof; A⁶ is selected from the group consisting of histidine, arginine, lysine, and a derivative thereof; A⁷ is selected from the group consisting of proline, glycine, serine, and a derivative thereof; and A⁸ can be present or absent, wherein when A⁸ is present, A⁸ is selected from the group consisting of serine, threonine, hydroxyproline, and a derivative thereof, provided (i) at least one of A¹-A⁸ is optionally substituted with a mono- or di-carbohydrate; or (ii) when A⁸ is absent: (a) at least one of A¹-A⁷ is substituted with a mono- or di-carbohydrate, (b) A⁷ is terminated with an amino group, or (c) a combination thereof.

In some embodiments, A¹ is the amino terminal end of the oligopeptide and A⁸ (or A⁷ when A⁸ is absent) is the carboxyl terminal end. Still in other embodiments, A¹ is the carboxyl terminal end and A⁸ (or A⁷ when A⁸ is absent) is the amino terminal end. Yet in other embodiments, the carboxylic acid functional group of the carboxyl terminal end is modified as an amide functional group, an amine functional group, a hydroxyl functional group, or a thiol functional group. The amide and the amine functional groups can be non-alkylate, mono-alkylated or di-alkylated.

Yet in other embodiments, the carbohydrate comprises glucose, galactose, xylose, fucose, rhamnose, or a combination thereof. In some instances, the carbohydrate is a mono-carbohydrate, whereas in other instances, the carbohydrate is a di-carbohydrate.

In other embodiments, at least one of A¹-A⁸ is substituted with a mono-carbohydrate. Still in other embodiments, at least one of A¹-A⁸ is substituted with a di-carbohydrate. It should be appreciated that the scope of the invention also includes those oligopeptides having both mono- and di-carbohydrates.

Exemplary di-carbohydrates that can be used in oligopeptides of the invention include, but are not limited to, lactose, cellobiose, melibiose, and a combination thereof. However, it should be appreciated that the scope of the invention includes oligopeptides that are substituted with any dicarbohydrates known to one skilled in the art.

In one particular embodiment, A⁸ is serine or a derivative thereof. In some instances, the carboxylic acid moiety of the serine is modified as an amide or an amine. In one case, serine is terminated as an amino group. Still in other embodiments, the serine residue of A⁸ is glycosylated with glucose or lactose.

Yet in other embodiments, at least one, typically at least two, generally at least three, often at least four, still more often at least five, yet still more often at least six, and most often all of A¹-A⁸ is D-amino acid.

Another aspect of the invention provides oligopeptides, such as Ang-(1-7) derivatives, having eight amino acids or less, typically seven or eight amino acid residues. In some embodiments, one or more amino acids have attached thereto a carbohydrate group. Often the carbohydrate group is attached to the oligopeptide via glycosylation. The carbohydrate can be attached to the oligopeptide via any of the side chain functional group of the amino acid or the amide group. Accordingly, the scope of the invention includes, but is not limited to, 0-glycosylate, N-glycosylate, S-glycosylated oligopeptides. The term “X-glycosylated” refers to having a carbohydrate attached to the oligopeptide via the heteroatom “X” of the amino acid. For example, for serine whose side-chain functional group is hydroxyl, “O-glycosylated” means the carbohydrate is attached to the serine's side-chain functional group, i.e., the hydroxyl group. Similarly, “N-glycosylation” of leucine refers to having the carbohydrate attached to the amino side-chain functional group of leucine. Typically, the glycosylation is on the side-chain functional group of the amino acid.

In some embodiments, the Ang-(1-7) derivative is glycosylated with xylose, fucose, rhamnose, glucose, lactose, cellobiose, melibiose, or a combination thereof.

Yet in other embodiments, the carboxylic acid terminal end of said glycosylated Ang-(1-7) derivative is substituted with an amino group. When referring to the carboxyl acid terminal end being substituted with an amino group, it means —OH group of the carboxylic acid is replaced with —NH₂ group. Thus, the actual terminal end functional group is an amide, i.e., rather than having the oligopeptide being terminated at the carboxylic acid terminal end with a functional group —CO₂H, the carboxylic acid terminal end is terminated with an amide group (i.e., —CO₂NR′2, where each R′ is independently hydrogen or C₁-C₁₂ alkyl). Still in other embodiments, the carboxylic acid terminal group is terminated with a hydroxyl or a thiol group. In some embodiments, the modified carboxylic acid terminal group is used to attach the carbohydrate, e.g., via glycosylation.

One of the purposes of the invention was to produce Ang-(1-7) derivatives to enhance efficacy of action, in vivo stabilization, and/or penetration of the blood-brain barrier. Improved penetration of the blood-brain barrier facilitates cerebral entry of the Ang-(1-7) derivative of the invention, and, consequently, Mas activation, or intrinsic-efficacy. To improve (i.e., increase) penetration of the blood-brain barrier, in some embodiments the Ang-(1-7) derivative is attached to at least one mono- or di-carbohydrates.

Without being bound by any theory, it is believed that the oligopeptide of the invention that are glycosylated exploits the inherent amphipathicity of the folded Ang-(1-7) glycopeptides (i.e., glycosylated oligopeptides of the invention) and the “biousian approach” to deliver the glycosylated oligopeptides of the invention across the blood-brain barrier. In some instances, the amount of increase in crossing the blood-brain barrier by oligopeptides of the invention is at least 6%, typically at least 10%, and often at least 15% compared to native Ang-(1-7). In some instances, the amount of increase in the Cmax for oligopeptides of the invention in cerebral-spinal fluid is 2-10 fold, 3-8 fold, or 5-8 fold compared to native Ang-(1-7). In some instances, the amount of increase in the Cmax for oligopeptides of the invention in cerebral-spinal fluid is 2, 3, 4, 5, 6, 7, 8, 9 or 10 fold compared to native Ang-(1-7). In other instances, oligopeptides of the invention have in vivo half-life of at least 20 min, at least 30 min, at least 40 min, at least 50 min, at least 60 min, or at least 2, hours, at least 3 hours, at least 4 hours, at least 5 hours or at least 6 hours. In some instances, the amount of increase in the in vivo half-life for oligopeptides of the invention is 2-30 fold, 3-25 fold, 4-20 fold, 4-10 fold, 10-25 fold, 15-25 fold, or 20-25 fold compared to native Ang-(1-7). Alternatively, compared to native Ang-(1-7), oligopeptides of the invention exhibit at least 50 fold, typically at least 75 fold, and often at least 100 fold increase in in vivo half-life.

In other embodiments, oligopeptides of the invention exhibit enhanced vascular efficacy. Without being bound by any theory, it is generally recognized that blood-brain barrier transport occurs via an absorptive endocytosis process on the blood side of the endothelium of the brain capillaries followed by exocytosis on the brain side, leading to overall transcytosis. It is also believed that for this process to be efficient, the oligopeptide must bind to the membrane for some period of time, and must also be able to exist in the aqueous state for some period of time (biousian nature). Based on previous work from one of the present inventors, it is believed that effective drug delivery and blood-brain barrier transport requires a biousian glycopeptide that has at least two states: (1) a state defined by one or more membrane-bound conformations that permit or promote endocytosis; and (2) a state defined by a water-soluble, or random coil state that permits “membrane hopping” and, presumably, vascular efficacy.

In general, the degree of glycosylation does not have a large effect on the structure of the individual microstates. Thus, altering the degree of glycosylation allows for the modulation of aqueous vs. membrane-bound state population densities without significantly affecting the overall structure of the oligopeptide. Moreover, it is believed that glycosylation also promotes stability to peptidases, thereby increasing the half-life of the Ang-(1-7) derivatives in vivo.

TABLE 1 sets forth some particularly useful Ang(1-7) derivative polypeptides but is not intended to be limiting on the scope of the invention.

| Amino Acid Position | | | | | | | | SEQ |
|---|---|---|---|---|---|---|---|---|
| 1 | 2 | 3 | 4 | 5 | 6 | 7 | 8 | ID NO: |
| Asp | Arg | Val | Tyr | Ile | His | Pro | — | 2 |
| Asp | Arg | Val | Tyr | Ile | His | Proº | — | 3 |
| Asp | Arg | Val | Tyr | Ile | His | Pro* | — | 4 |
| Asp | Arg | Val | Tyr | Ile | His | Proº* | — | 5 |
| Asp | Arg | Val | Tyr | Ile | His | Pro | Ser | 6 |
| Asp | Arg | Val | Tyr | Ile | His | Pro | Serº | 7 |
| Asp | Arg | Val | Tyr | Ile | His | Pro | Ser* | 8 |
| Asp | Arg | Val | Tyr | Ile | His | Pro | Serº* | 9 |
| Asp | Arg | Val | Tyr | Ile | His | Ser | — | 10 |
| Asp | Arg | Val | Tyr | Ile | His | Serº | — | 11 |
| Asp | Arg | Val | Tyr | Ile | His | Ser* | — | 12 |
| Asp | Arg | Val | Tyr | Ile | His | Serº* | — | 13 |
| Ala | Arg | Val | Tyr | Ile | His | Pro | — | 14 |
| Ala | Arg | Val | Tyr | Ile | His | Proº | — | 15 |
| Ala | Arg | Val | Tyr | Ile | His | Pro* | — | 16 |
| Ala | Arg | Val | Tyr | Ile | His | Proº* | — | 17 |
| Ala | Arg | Val | Tyr | Ile | His | Pro | Ser | 18 |
| Ala | Arg | Val | Tyr | Ile | His | Pro | Serº | 19 |
| Ala | Arg | Val | Tyr | Ile | His | Pro | Ser* | 20 |
| Ala | Arg | Val | Tyr | Ile | His | Pro | Serº* | 21 |
| Ala | Arg | Val | Tyr | Ile | His | Ser | — | 22 |
| Ala | Arg | Val | Tyr | Ile | His | Serº | — | 23 |
| Ala | Arg | Val | Tyr | Ile | His | Ser* | — | 24 |
| Ala | Arg | Val | Tyr | Ile | His | Serº* | — | 25 |
| Asp | Arg | Nle | Tyr | Ile | His | Pro | — | 26 |
| Glu | Lys | Val | Ser | Val | Arg | Ser | | |
| Ala | Ala | Leu | Thr | Leu | — or º | Cys | | |
| Asn | — or º | Ile | Ala | Nle | | —, º, *, | | |
| Pro | | Ala | — or º | Ala | | or º* | | |
| Gly | | Gly | | Gly | | | | |
| — or º | | Lys | | | | | | |
| | | Pro | | | | | | |
| | | Tyr | | | | | | |
| | | — or º | | | | | | |
| Asp | Arg | Nle | Tyr | Ile | His | Pro | Phe | 27 |
| Glu | Lys | Val | Ser | Val | Arg | Ala | Ser | |
| Ala | Ala | Leu | Thr | Leu | — or º | | Cys | |
| Asn | — or º | Ile | Ala | Nle | | | Ile | |
| Pro | | Ala | — or º | Ala | | | Tyr | |
| Gly | | Gly | | Gly | | | —, º, *, | |
| — or º | | Lys | | | | | or º* | |
| | | Pro | | | | | | |
| | | Tyr | | | | | | |
| | | — or º | | | | | | |

¹ - Where more than one amino acid is indicated, the amino acids are presented in the alternative.
— = unmodified
º = glycosylated
* = carboxy terminal NH₂

In some embodiments, only the C-terminal amino acid is glycosylated (i.e., Xaa⁸ or Xaa⁷ if Xaa⁸ is absent). In some embodiments, the Ang(1-7) derivative polypeptide is glycosylated with glucose, lactose, cellobiose, melibiose, β-D-glucose, β-D-lactose, β-D-cellobiose, or β-D-melibiose. In some embodiments, the polypeptide comprises an O-linked glycosylation (e.g., on the R-group of a serine). In some embodiments, the C-terminal serine is glycosylated.

In some embodiments, non-naturally-occurring amino acids and/or amino acid substitutes (e.g., dicarboxylic acids) may be substituted for the naturally-occurring amino acids in Ang(1-7) and any of the Ang(1-7) derivative polypeptides including, for example, in the Ang(1-7) derivative polypeptides of TABLE 1. For example, α,α-disubstituted amino acids, N-alkyl amino acids, C-α-methyl amino acids, β-amino acids, and β-methyl amino acids. Amino acids analogs useful in the present invention may include, but are not limited to, β-alanine, norvaline, norleucine, 4-aminobutyric acid, orithine, hydroxyproline, sarcosine, citrulline, cysteic acid, cyclohexylalanine, 2-aminoisobutyric acid, 6-aminohexanoic acid, t-butylglycine, phenylglycine, o-phosphoserine, N-acetyl serine, N-formylmethionine, 3-methylhistidine and other unconventional amino acids. For example,
Xaa¹ may be Acpc (1-aminocyclopentane carboxylic acid), Me2Gly (N,N-dimethylglycine), Bet (betaine, l-carboxy-N,N,N-trimethylmethanaminium hydroxide), Sar (sarcosine) or Suc (succinic acid);
Xaa² may be Cit (citrulline), Orn (ornithine), acetylated Ser, or Sar;
Xaa³ may be Nle (norleucine), hydroxyproline, Acpc, or Aib (2-aminoisobutyric
acid);
Xaa⁴ may be Tyr(PO₃), homoserine, azaTyr (aza-α¹-homo-L-tyrosine);
Xaa⁵ may be Nle, hydroxyproline, Acpc, or Aib;
Xaa⁶ may be 6-NH₂-Phe (6-aminophenylalaine): and
Xaa⁸ may be Phe(Br) (p-bromo-phenylalanine; may be L- or D-phenylalanine).

In some embodiments, the Ang(1-7) derivative polypeptide does not comprise the naturally-occurring amino acid sequence of native Ang(1-7) set forth in SEQ ID NO: 2.

In some embodiments, Ang(1-7) and any of the Ang(1-7) derivative polypeptides, including those specifically defined in TABLE 1, may comprise entirely L-amino acids, entirely D-amino acids, or a mixture of L- and D-amino acids (e.g., having 1, 2, 3, 4, 5, 6, 7, or 8 D-amino acids).

The Ang(1-7) and Ang(1-7) derivative polypeptides may be produced by any suitable method including, without limitation, by peptide synthesis methods such exclusive solid phase synthesis, partial solid phase synthesis, fragment condensation, classical solution synthesis, native-chemical ligation, and recombinant techniques.

Cognitive Dysfunction

Cognitive dysfunction or impairment is a common neurological complication of congestive heart failure (“CHF”) and post cardiac surgery affecting approximately 50-70% of patients at hospital discharge and 20-40% of patients six months after surgery. The occurrence of CHF and postoperative cognitive dysfunction is associated with increased duration of hospitalization and impaired long-term quality of life. Without being bound by any theory, it is believed that in general any clinical condition associated with an increase in inflammatory cytokines and/or increase in reactive oxygen species in central nervous system, in particular in the brain, can lead to cognitive dysfunction.

Other aspects of the invention provide methods for treating cognitive dysfunction and/or impairment in a patient using an oligopeptide of the invention. Typically, methods of the invention include administering to a patient in need of such a treatment a therapeutically effective amount of an oligopeptide of the invention. It should be appreciated that the oligopeptides of the invention can be used to treat any clinical conditions that are known to be treatable or appears to be treatable using Ang-(1-7). However, for the sake or clarity and brevity, the invention will now be described in reference to treating cognitive dysfunction and/or impairment in a patient.

The cognitive dysfunction that occurs in congestive heart failure (CHF) patients includes decreased attention, memory loss, psychomotor slowing, and diminished executive function, all of which compromises patients' ability to comply with complex medical regimens, adhere to dietary restrictions and make self-care decisions. Mechanisms thought to contribute to cognitive impairment in patients with CHF include changes in cerebral blood flow, altered cerebrovascular autoregulation and microembolisms. In one study, cerebral blood flow was measured with single-photon emission computed tomography (SPECT) and found to be reduced by 30% in patients with severe heart failure. The causes for decreased cerebral perfusion in CHF have been attributed to low cardiac output, low blood pressure and altered cerebrovascular reactivity. In some cases, the cognitive impairment seen in CHF is improved following either heart transplant or improvement in cerebral blood flow via optimal management of CHF. However, for many patients with CHF, management is rarely optimal, and the cognitive impairment persists. Interestingly, long-term follow up studies have revealed that cognitively normal CHF patients have a significantly higher risk of dementia or Alzheimer's disease compared to age-matched non-CHF controls, suggesting that CHF and cardiovascular disease predispose patients to further cognitive impairment and dementia.

During CHF, the well characterized changes in the circulating neurochemical milieu and increases in inflammatory factors are also seen in the brain. Most of the studies on CHF-induced changes in inflammatory cytokines and ROS have focused on brain regions involved in sympathetic outflow regulation and not on cognition. CHF elevates sympathetic tone and causes abnormal cardiac and sympathetic reflex function. In the rat, ischemia-induced CHF significantly increases pro-inflammatory cytokines and Angiotensin II type 1 receptors (AT1) in the paraventricular nucleus (PVN) of the hypothalamus. Further, in CHF rabbits, the increase in sympathetic outflow is blocked by ICV injection of the super oxide dimustase (SOD) mimetic tempol, presumably by inhibition of ROS. CHF in this model is associated with increased expression of NADPH oxidase subunits and ROS production in the rostral ventral lateral medulla (RVLM) and increases in NO.

The role of ROS in learning and memory has been extensively studied. All of the NAD(P)H oxidase subunits, including NOX2 and NOX4, have been localized within the cell bodies and dendrites of neurons of the mouse hippocampus and perirhinal cortex and are co-localized at synaptic sites. These are key regions of the brain in learning and memory. In the brain, superoxide production via actions of NAD(P)H oxidase are known to be involved in neurotoxicity, age related dementia, stroke and neurodegenerative diseases and have been identified throughout the brain including the hippocampus, thalamus, cerebellum and amygdala. In younger, healthy animals ROS and NAD(P)H oxidase is shown to be required for normal learning and hippocampal long-term potentiation (LTP). Recent studies in mice lacking Mas have shown that Ang-(1-7) and Mas are essential for normal object recognition processing and blockade of Mas in the hippocampus impairs object recognition. In addition, Ang-(1-7) facilitates LTP in CA1 cells and this effect is blocked by antagonism of Mas. In older animals or in CHF animals, an increase in ROS is linked to LTP and memory impairments.

Over the last decade, it has become recognized that renin angiotensin system (RAS) involves two separate enzymatic pathways providing a physiological counterbalance of two related peptides acting at distinct receptors. The well described ACE-Angll-AT1 receptor system is thought to be physiologically opposed and balanced by the ACE2-Ang-(1-7)-Mas system. Functionally, these two separate enzymatic pathways of RAS are thought to be involved in balancing ROS production and nitric oxide (NO) in the brain, microvasculature and peripheral tissues. Increases in AT1 receptor activation are known to increase NAD(P)H oxidase and ROS generation which are both known to contribute to abnormal increases of sympathetic nerve activity observed in CHF and hypertension. This increase in AT1 receptor-induced ROS formation is thought to be opposed by ACE2-Ang-(1-7)-Mas inhibition of ROS formation. Ang-(1-7), the majority of which is produced from ACE2 cleavage of Ang II, decreases ROS production and increases NOS in the brain via activation Mas and, possibly through AT2 receptor.

Within the brain, the Mas receptor is known to be expressed on neurons, microglia and vascular endothelial cells. Further, all three of these key components that make up the “neurovascular unit” (neurons, microglia and endothelial cells) are central players in neurogenic hypertension and CHF-induced increases in brain inflammation and ROS production. Both CHF and hypertension increase circulating cytokines promoting ROS production within the “neurovascular unit”. The end-result of this feed-forward cascade is neuronal dysfunction and cognitive impairment. The ideal therapeutic candidate to treat cognitive impairment would be designed to interrupt this cascade by working at both sides of the blood-brain barrier, the brain vascular endothelium and neuronal cells. Ang-(1-7), acting at the Mas receptor, is known to have effects at both endothelial cells and neurons. However, using a native Ang-(1-7) for treating cognitive dysfunction and/or impairment is not suitable because native Ang-(1-7) is susceptible to enzymatic degradation. Moreover, native Ang-(1-7) does not readily cross the blood-brain barrier to be suitable as a therapeutic agent.

Without being bound by any theory, it is believed that one of the advantages of using oligopeptides of the invention in treating cognitive dysfunction and/or impairment is that oligopeptides of the invention have enhanced endothelial “interaction” and brain penetration. It is believed that oligopeptides of the invention act at both endothelial cells and neurons thus inhibiting inter alia neurovascular ROS production and mitigating the brain inflammatory cascade.

Accordingly, oligopeptides the invention can be used to treat cognitive impairment and/or dysfunction (1) associated with pre- and/or post-surgery dementia, or (2) observed in patients with congestive heart failure, cardiovascular disease, or hypertension. More generally, oligopeptides of the invention are useful in treating cognitive dysfunction and/or impairment in a subject whose cognitive dysfunction and/or impairment is clinically associated with an increase in inflammatory cytokines and/or increase in reactive oxygen species (“ROS”) in the central nervous system, in particular the brain. As used herein, the term “clinically associated” refers to the root cause or underlying cause of cognitive dysfunction and/or impairment (such as, but not limited to, memory loss) that when ameliorated results in reduction, prevention, treatment or reversal of cognitive dysfunction and/or impairment. Exemplary clinical conditions associated with an increase in inflammatory cytokines and/or increase in reactive oxygen species that can cause cognitive dysfunction and/or impairment include, but are not limited to, circulatory compromise, cardiovascular disease, hypertension, hypotension, congestive heart failure, stroke, embolism, surgery (e.g., postoperative recovery condition), dementia, Alzheimer's disease, disease related cognitive impairment, trauma related cognitive impairment, age-related dementia, postoperative related delirium and/or increase in inflammatory cytokine and/or increase in reactive oxygen species within the central nervous system of said subject or a combination thereof.

Methods of Administration

Oligopeptides of the present invention can be administered to a patient to achieve a desired physiological effect. Preferably the patient is an animal, more preferably a mammal, and most preferably a human. The oligopeptide can be administered in a variety of forms adapted to the chosen route of administration, i.e., orally or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic.

The active oligopeptide can be orally administered, for example, with an inert diluent or with an assimilable edible carrier, or it can be enclosed in hard or soft shell gelatin capsules, or it can be compressed into tablets. For oral therapeutic administration, the active oligopeptide may be incorporated with excipient and used in the form of ingestible tablets, buccal tablets, troches, capsules, elixirs, suspensions, syrups, wafers, and the like. Such compositions and preparation can contain at least 0.1% of active oligopeptide. The percentage of the compositions and preparation can, of course, be varied and can conveniently be between about 1 to about 10% of the weight of the unit. The amount of active oligopeptide in such therapeutically useful compositions is such that a suitable dosage will be obtained. Preferred compositions or preparations according to the present invention are prepared such that an oral dosage unit form contains from about 1 to about 1000 mg of active oligopeptide.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active oligopeptide, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active oligopeptide can be incorporated into sustained-release preparations and formulation.

The active oligopeptide can also be administered parenterally. Solutions of the active oligopeptide can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the active oligopeptide in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The therapeutic oligopeptides of the present invention can be administered to a mammal alone or in combination with pharmaceutically acceptable carriers, as noted above, the proportion of which is determined by the solubility and chemical nature of the oligopeptide, chosen route of administration and standard pharmaceutical practice.

The physician will determine the dosage of the present therapeutic agents which will be most suitable for prophylaxis or treatment and it will vary with the form of administration and the particular oligopeptide chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Example 1: Ang-(1-7) Derivative High-Throughput Screening (HTS)

For HTS, a sensitive and direct measure of nitric oxide (NO) production in 2 separate cell lines is utilized, primary CA1 hippocampal neurons and human umbilical vein endothelial cells (HUVEC). The use of primary CA1 cells is self-evident for the study of central effects. In addition, the contribution of endothelial dysfunction to the progression of CHF and to the induction of cognitive impairment is clinically appreciated. The emerging picture that the Ang-(1-7) singling axis holds promise as a therapeutic target for endothelial dysfunction strongly indicates that reversal of CHF-induced endothelial dysfunction as mechanism cannot be ruled out. HUVEC are isolated from the human umbilical vein and cryo-preserved after primary culture. HUVEC is included as a second line for the primary screen because these cells are the model in vitro system for the study of endothelial cell function and can be used to directly measure Mas-dependent NO production.

Cell Culture.

To isolate primary hippocampal CA1 neuronal cells, whole brain was removed from neonatal rat pups (1-2 day old) and the cortices dissected away. The hippocampus was isolated and the CA1 field was excised and placed in buffer. After gentle disruption in digestion buffer, the cells were counted, placed in culture media, and plated in a 96-well format coated with poly-d-lysine. At the time of plating, cells were approximately at 50% density and were allowed to culture to 70-80% density before starting the assay. Commercially available HUVEC (Life Technologies/Thermo Fisher) was thawed and plated (5000-10,000 cells/well) in a 96-well format coated with gelatin. HUVEC cells were allowed to culture overnight before starting the assay.

Cell Activation:

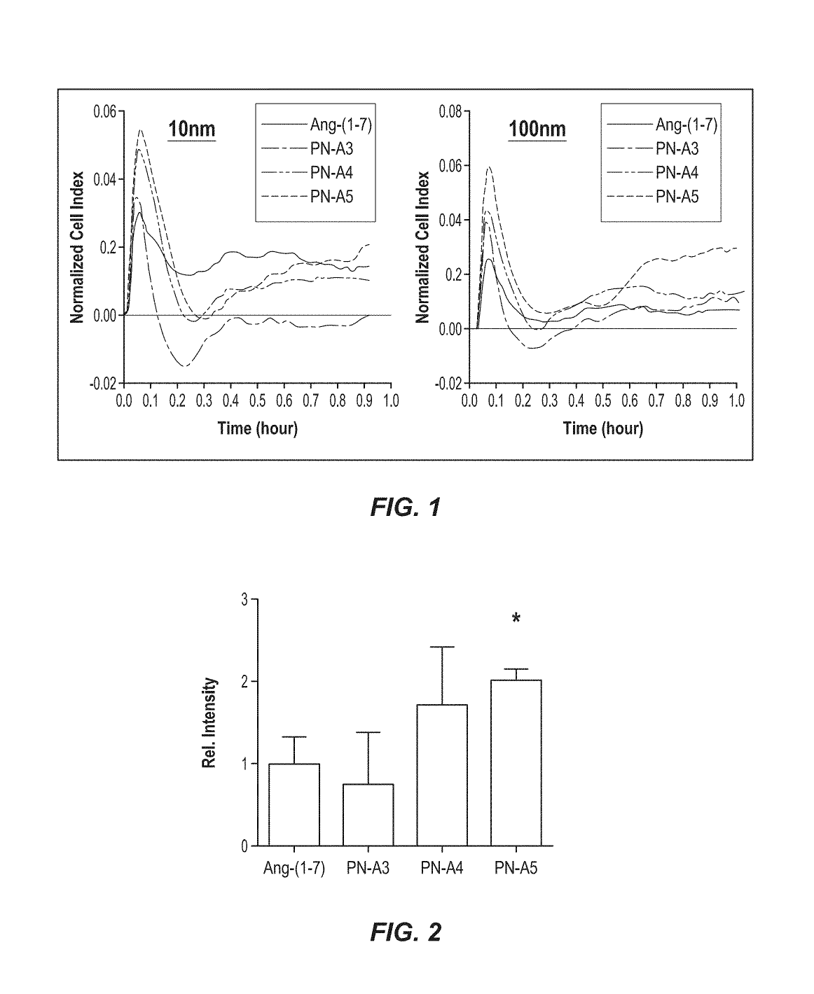

The xCELLigence system Real-Time Cell Analyzer (RTCA), developed by Roche Applied Science, uses microelectronic biosensor technology to do dynamic, real-time, label-free, and non-invasive analysis of cellular events including G-protein receptor activation of cells. The RTCA analysis was utilized to measure the potency and relative ability of oligopeptides of the invention and native Ang-(1-7) to activate human umbilical vascular endothelial cells (HUVEC) in culture. Following uniform cellular adherence based on a linear increase in cell impedance (CI), HUVECs were treated with Ang-(1-7) and oligopeptides of the invention. Each trace of the CI over time in FIG. 1 represents the average of 4 wells normalized to CI at the time of compound addition. FIG. 1 shows the results from data acquired using the xCELLigence RTCA to measure the relative potency of PN-A3, PN-A4, PN-A5 and native Ang-(1-7). A 100 nM administration of PN-A3, PN-A4 and PN-A5 and 10 nM of PN-A3 and PN-A5 resulted in a significant (˜2-fold) increase in CI over the native Ang-(1-7) demonstrating that the oligopeptides of the invention have greater potency for cell activation than native Ang-(1-7).

NO Production Assay.

As a screen for mechanisms of action of oligopeptides of the invention, the ability to increase NO production of three oligopeptides of the invention (PN-A3, PN-A4 and PN-A5) were characterized and compared to native Ang-(1-7). Human umbilical vascular endothelial cells (HUVEC) culture plates received fluorescence reaction buffer (0.2 M phosphate buffer, pH 7, 1 mM EDTA, 0.1% glucose) containing diaminofluorescein-FM diacetate (DAF-FM, 1 μM) to measure real-time NO production. Time-resolved (10 minutes) fluorescent intensity was detected using a BioTek Synergy 2 microplate reader with excitation at 485 nm and emission at 535 nm. DAF-FM is a sensitive flourometric derivative for the selective detection of NO in live cells.

FIG. 2 shows relative peak fluorescence intensity following 5 minutes exposure to native Ang-(1-7) and three oligopeptides of the invention. Values were normalized to control fluorescence. As expected, native Ang-(1-7) induced a significant elevation of NO over control levels. More importantly, as shown in FIG. 2, oligopeptides of the invention (namely PN-A3, PN-A4 and PN-A5) also elicited a significant elevation of NO over control levels, with PN-A5 significantly enhancing NO production over that seen with native Ang-(1-7), *=p<0.05. These results demonstrate that oligopeptides of the invention increase NO production similar to or greater than that of native Ang-(1-7).

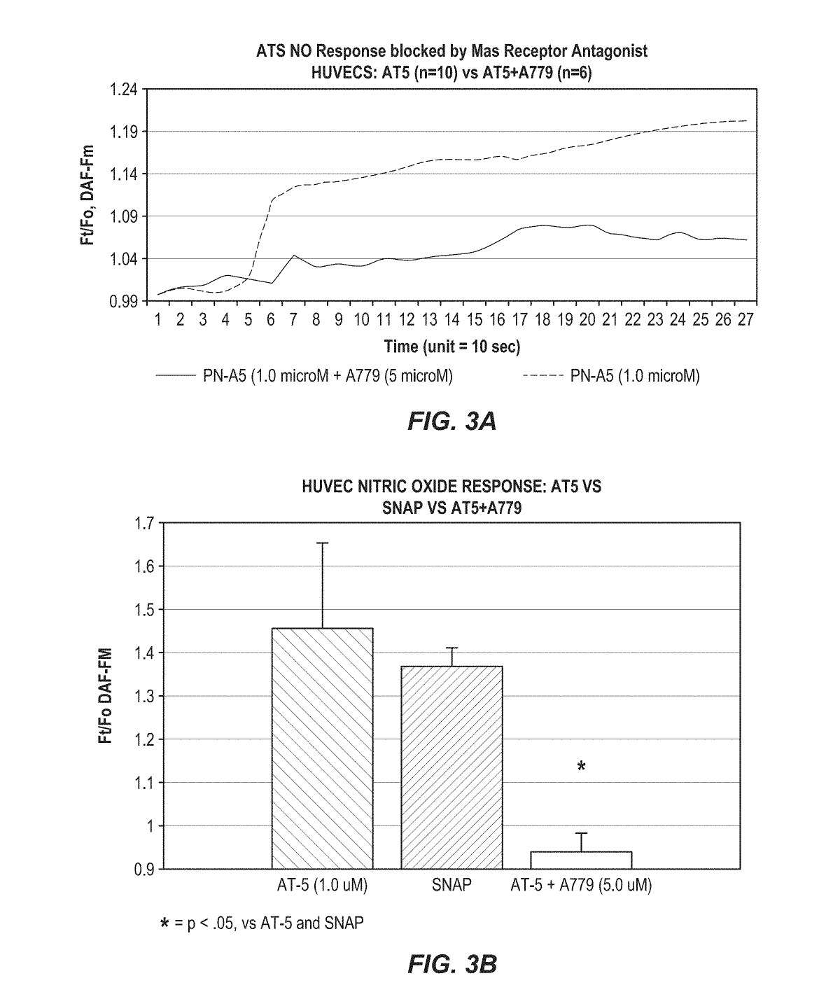

FIG. 3A illustrates the ability of the select Mas receptor antagonists, A779, (C₃₉H₆₀N₁₂O₁₁) which is known to block native Ang-(1-7) NO production, to also block NO production induced by the oligopeptide of the invention, namely PN-A5. In these studies, HUVEC cells were incubated with DAF-FM, 1 μM to measure real-time NO production. Cells were treated with either PN-A5 alone (1.0 mM, n=10), PN-A5+A779 (n=6). Measurements were obtained using an Olympus 550 Confocal Microscope and analyzed using Image J. Images were obtained every 10 sec. These results indicate that the oligopeptide PN-A5 actions are due to activation of the Mas receptor.

FIG. 3B shows the averaged effect of the select Mas receptor antagonists, A779, which is known to block native Ang-(1-7) NO production, to also block NO production induced by the oligopeptide of the invention, PN-A5. In these studies, HUVEC cells were incubated with DAF-FM, 1 μM to measure real-time NO production. Cells were treated with either PN-A5 alone (1.0 mM, n=10), PN-A5+A779 (n=6), or the NO donor S-nitroso-N-acetylpenicillamine (SNAP). Fluorescent measurements were obtained using an Olympus 550 Confocal Microscope and analyzed using Image J. Images were obtained every 10 sec. The NO response produced by PN-A⁵ was completely blocked by A779 demonstrating that PN-A5's ability to increase NO is due to PN-A5 actions on the Mas receptor.

### Example 2: Effects of Ang-(1-7) Derivative on Heart Failure (HF) Induced Cognitive Impairment

A total of 33, male C57Bl/6J adult mice (Harlan, 8-10 weeks old) were used. Mice were randomly assigned to either the sham (n=12) or congestive heart failure (CHF) group (n=21). Experimental groups are described as follows: sham+saline, CHF+saline, CHF+PN-A5. All mice prior to surgery were weighed and anesthetized. For the CHF mice, MI was induced by ligation of the left coronary artery (LCA). Under anesthesia (2.5% isoflurane in a mixture of air and O₂) a thoracotomy was performed at the fourth left intercostal space and the LCA permanently ligated to induce a myocardial infarction (MI). Occlusion of the LCA was confirmed by observing blanching, a slight change in color of the anterior wall of the left ventricle downstream of the ligature. Sham mice underwent the same procedure with the exception of ligating the LCA.

Following 8 weeks post MI surgery, CHF mice were treated with either daily subcutaneous injections of the Ang-(1-7) derivative PN-A5 (1 mg/kg/day) for 28 days or saline. After 21 days, animals were tested for object recognition using a standard NOR test as described below. After approximately 25 days of treatment, animals were tested for spatial memory using the standard Morris water task as described below.

Novel Object Recognition (NOR):

The apparatus consisted of an evenly illuminated Plexiglas box (12 cm×12 cm×12 cm) placed on a table inside an isolated observation room. All walls of the apparatus were covered in black plastic, and the floor was grey with a grid that was used to ensure that the location of objects did not change between object familiarization and test phases. The mouse behavior and exploration of objects was recorded with a digital camera. The digital image from the camera was fed into a computer in the adjacent room. Two digital stopwatches were used to track the time the mouse spent interacting with the objects of the test. All data was downloaded to Excel files for analysis. Triplicate sets of distinctly different objects were used for the test.

The novel object recognition task included 3 phases: habituation phase, familiarization phase, and test phase. For the habituation phase, on the first and second day, mice were brought to the observation room habituated to the empty box for 10 min per day. On the third day, each mouse had a “familiarization” trial with two identical objects followed by a predetermined delay period and then a “test” trial in which one object was identical to the one in the familiarization phase, and the other was novel. All stimuli were available in triplicate copies of each other so that no object needed to be presented twice. Objects were made of glass, plastic or wood that varied in shape, color, and size. Therefore, different sets of objects were texturally and visually unique. Each mouse was placed into the box the same way for each phase, facing the center of the wall opposite to the objects. To preclude the existence of olfactory cues, the entire box and objects were always thoroughly cleaned with 70% ethanol after each trial and between mice. During the familiarization phase, mice were allowed to explore the two identical objects for 4 min and then returned to their home cages. After a 2 hour delay, the “test phase” commenced. The mice were placed back to the same box, where one of the two identical objects presented in the familiarization phase was switched to a novel one and the mouse was allowed to explore these objects for another 4 min. Mouse “exploratory behavior” was defined as the animal directing its nose toward the object at a distance of ˜2 cm or less. Any other behavior, such as resting against the object, or rearing on the object was not considered to be exploration. Exploration was scored by an observer blind to the mouse's surgical group (CHF vs. Sham). Finally, the positions of the objects in the test phases, and the objects used as novel or familiar, were counterbalanced between the 2 groups of mice.

Discrimination ratios were calculated from the time spent exploring the novel object minus time spent exploring the familiar object during the test phase divided by the total exploration time. DRatio=(t novel−t familiar)/(t novel+t familiar). Data were analyzed from first 2 minutes of ‘test phase’. A positive score indicates more time spent with the novel object, a negative score indicates more time spent with the familiar object, and a zero score indicates a null preference. All NOR data was examined using one-way analysis of variance, between subjects (ANOVA). Individual group differences were tested using the post hoc Tukey HSD test. In comparisons between groups of different sample sizes, equal variance was tested using a modified Levene's test. All statistical tests and p-values were calculated using MS Excel with Daniel's XLtoolbox and alpha was set at the 0.05 level. Error bars represent SEM.

Morris Water Task: Testing Spatial Learning and Memory/Visual Test:

The apparatus used was a large circular pool approximately 1.5 meters in diameter, containing water at 25° C. made opaque with addition of non-toxic white Crayola paint. An escape platform was hidden just below the surface of the water. Visual, high contrast cues were placed on the walls of the test room. A digital camera connected to a computer in the adjacent room is suspended over the tank to record task progress. For spatial testing prior to MI at 4 and 8 weeks post-MI or sham surgery, the platform was located at different sites in the pool.

During the spatial version of the Morris water task, all animals were given 6 training trials per day over 4 consecutive days. During these trials, an escape platform was hidden below the surface of water. Mice were released from seven different start locations around the perimeter of the tank, and each animal performed two successive trials before the next mouse was tested. The order of the release locations was pseudo-randomized for each mouse such that no mouse was released from the same location on two consecutive trials. Performance on the swim task was analyzed with a commercial software application (ANY-maze, Wood Dale, Ill.). Because different release locations and differences in swimming velocity produce variability in the latency to reach the escape platform, a corrected integrated path length (CIPL) was calculated to ensure comparability of mice performance across different release locations. The CIPL value measures the cumulative distance over time from the escape platform corrected by an animal's swimming velocity, and is equivalent to the cumulative search error. Therefore, regardless of the release location, if the mouse mostly swims towards the escape platform the CIPL value will be low. In contrast, the more time a mouse spends swimming in directions away from the platform, the higher the CIPL value.

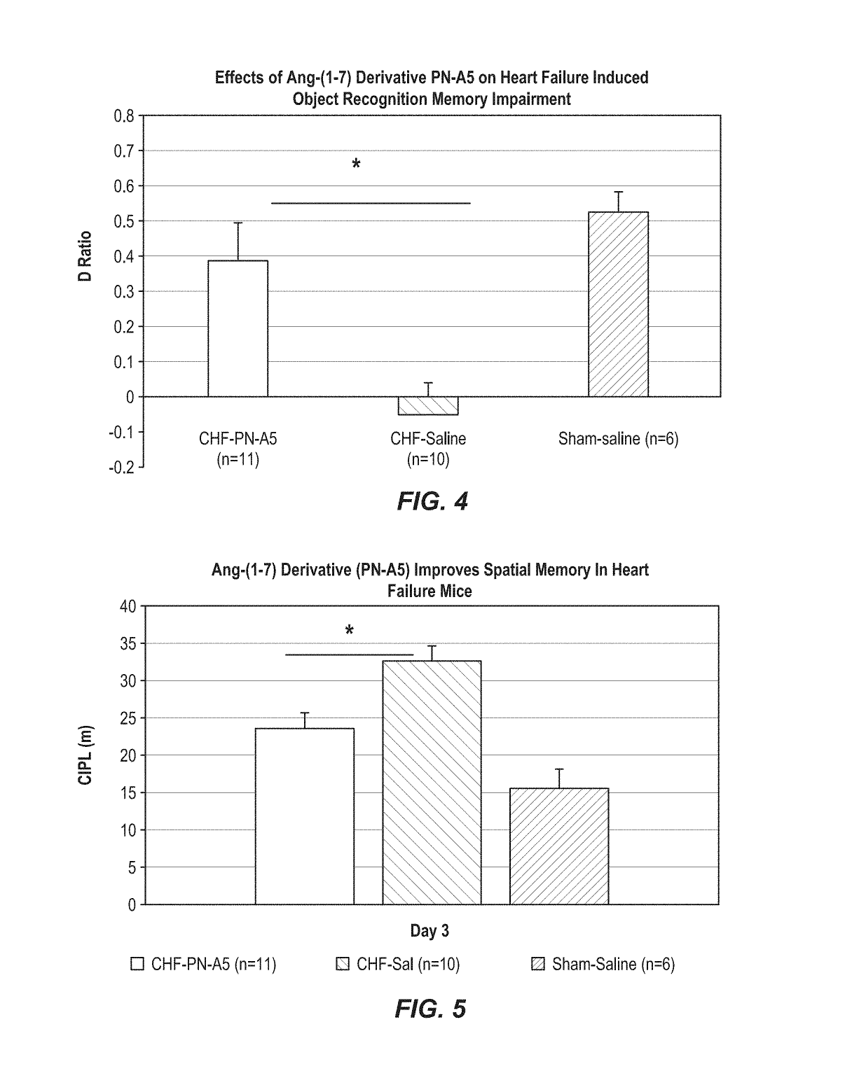

Following approximately 21 days of treatment with oligopeptide PN-A5, CHF mice showed object recognition memory improvement. FIG. 4 illustrates the effects of three weeks treatment with oligopeptide PN-A5 on object recognition memory as determined by the Novel Object Recognition Test (NOR). The mean performance of CHF mice with oligopeptide PN-A5 treatment (n=11) was similar to sham mice with saline (n=6), (CHF-Ang-(1-7) derivative PN-A5 M=+0.38, SE 0.11 vs. Sham-saline M=+0.52, SE 0.06) and significantly greater in comparison to CHF mice treated with saline (n=10) (M=−0.05, SE 0.09, *=p=0.009. These results demonstrate that oligopeptide PN-A5 acts to attenuate and even rescue object recognition memory impairment in mice with CHF.

Following approximately 25 days of treatment with oligopeptide PN-A5, CHF mice showed spatial memory improvement. FIG. 5 shows the mean CIPL of CHF+oligopeptide PN-A⁵ mice (n=11), CHF-saline treated mice (n=10) and Sham+saline mice (n=6). The CHF+oligopeptide PN-A5 mice showed significant improvement in spatial memory day 3 of the Morris swim task as compared to CHF-saline mice. CHF mice treated with saline had a significantly higher CIPL score as compared to CHF-oligopeptide PN-A5 treated mice (CHF-saline M=32.5, SE=2.1 vs CHF-oligopeptide PN-A5 M=23.5, SE 2.2, *=p=0.003. These results demonstrate that oligopeptide PN-A5 improves spatial memory.

### Example 3: Ang(1-7) Mitigates Cognitive Deficits Caused by Traumatic Brain Injury

Twenty-four C57/Bl6 mice (5.5 weeks, mass=18 to 20 g) were used for the duration of the study. The animals were housed in a humidity- and temperature-controlled environment and maintained on a 12:12 light:dark cycle (7:00 am-7:00 pm). Standard food and water were available ad libitum. The mice were divided into two main treatment groups: 1.) intraperitoneal (i.p.) injections of a normal saline (0.90%) vehicle (n=12) and (2.) i.p. injections of 0.1 mg/mL Ang-(1-7) (1 mg/kg) (n=12). A traumatic brain injury (TBI) model of closed head injury in mice using a pneumatic impactor capable of delivering a blow of a predetermined velocity, depth, and dwell time (duration of cortical depression) to a defined, 7.07 mm² area of the skull (Xiong, Mahmood, & Chopp, 2013) was used. Mice were first anesthetized using a 5% isoflurane vapor for induction. Once a response to toe-pinch was no longer observed, the mice were secured in the ear bars of a stereotaxic frame beneath the head impactor (TBI-0310 Impactor, Precision Systems) during which time 2.5% isoflurane was administered for maintenance of anesthesia. The parameters of each administered impact were set to the following: diameter of tip of cylindrical piston=3 mm; velocity of piston (vₚ)=4.0 m/s; depth of impact (dᵢ)=1 mm; dwell time (t_{dwell})=0.5 s. The point of impact was universalized in the medio-lateral plane to 1.5 mm left of the sagittal suture (as estimated by the mid-sagittal line of the mouse's head), and in the antero-posterior plane to an imaginary line intersecting the anterior point of insertion of the mouse's ears (approximately 1-2 mm anterior to the lambdoid suture). This point was chosen so as to avoid rupture of the superior sagittal sinus and the confluence of sinuses.

Immediately after being subjected to impact, mice were monitored for recovery of spontaneous respiration. Once noted to be breathing normally, mice were placed on the bedding of their normal enclosures and allowed to recover for 24 hours prior to their first, post-TBI novel object recognition trial.

The novel object recognition (NOR) task, as it pertains to the study of working memory and attention, is predicated on rodent preference of novel stimuli, whether spatial or otherwise (Ennaceur, Cavoy, Costa, & Delacour, 1989; Ennaceur & Delacour, 1988; Goulart et al., 2010; Silvers, Harrod, Mactutus, & Booze, 2007). When novel objects are paired simultaneously with familiar ones in an environment to which the animal has been habituated, it is possible to use the difference in exploration times of each object to make determinations of the degree of cognitive impairment relative to a measured baseline (Aggleton, Albasser, Aggleton, Poirier, & Pearce, 2010; Antunes & Biala, 2012; Olarte-Sanchez, Amin, Warburton, & Aggleton, 2015). The primary metric used to compare mice of different groups is the discrimination ratio (DR)—a value calculated as the ratio of time spent exploring the novel object (NO) to the total time spent exploring the familiar objects (FO) in addition the NO, i.e. DR=Time at NO/(Time at NO+Time at FO). In slight contrast to definitions of exploration used by previous authors (Aggleton et al., 2010; Aubele, Kaufman, Montalmant, & Kritzer, 2008; Ennaceur & Delacour, 1988; Goulart et al., 2010; Silvers et al., 2007), exploration in this investigation was defined as the directing of the nose toward an object at a distance of <2 cm from the object, touching an object with the nose or mouth, touching the object with both front paws, or standing on the object itself.

The overall structure of the NOR task, including associated familiarization trials, is as follows: mice from both groups underwent a two-day, combined habituation/familiarization phase, wherein they were allowed to roam freely in an evenly-lit, plastic, rectangular enclosure with walls 19.05 cm in height, containing three identical objects made of either glass or plastic, for five minutes. On the third day, the same test was run, but with one of the three “familiar” objects replaced with the NO, all spatial characteristics of the enclosure and objects therein remaining the same. Data collected on the third day constituted each mouse's baseline DR. On the fourth day, mice in both groups were subject to TBI as delineated in the previous section. The fifth day constituted the 24-hour post-TBI time point, wherein mice were administered an i.p. injection of either normal saline (vehicle group) or Ang-(1-7) solution (drug group) 30 minutes prior to undergoing the NOR task (NOs were rotated such that no animal saw the same NO twice). This pattern of injection and subsequent NOR trial was repeated to five days post-TBI. Both groups were run through two additional NOR tasks on post-TBI days 8 and 16 without prior drug or saline administration, again on post-TBI day 18 with prior drug or saline administration, and again on post-TBI day 25 without prior drug or saline administration. All NOR trials were filmed in high definition and manually reviewed using two stopwatches to determine the time spent at either a novel or familiar object.

Temporal data collected from each NOR trial was tabulated in a Microsoft Excel (2016) spreadsheet and individual discrimination ratio values calculated therein. Two-way ANOVA followed by a Tukey range test was performed using GraphPad Prism version 7.00 for Windows, GraphPad Software, La Jolla Calif. USA.

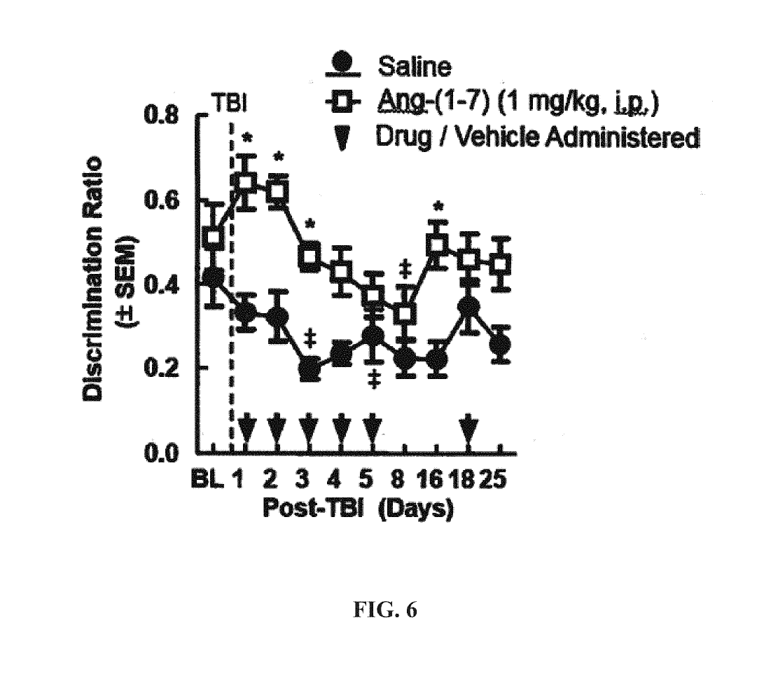

FIG. 6 provides the time course showing the development of the TBI-induced cognitive impairment. A baseline measure (“BL”) was obtained before TBI induction. As shown in FIG. 6, treatment with native Ang(1-7) significantly reduced the onset, severity, and duration of the TBI-induced cognitive impairment relative to vehicle controls.

### Example 4: Glycosylation of Ang(1-7) and its Derivatives Improves Pharmacokinetic Properties

One known limitation of therapeutically administering native Ang(1-7) is its relatively short half-life and relatively poor blood-brain-barrier permeability. The following experiments used a rational drug design approach to assess the effect of adding various glycosides to Ang(1-7) and its derivatives on serum half-life and BBB permeability. Stability in vivo is affected by a number of factors, including susceptibility to peptidases and glycosidases, as well as aggregation phenomena in solution, and a wide array of binding events, including membrane absorption. Interaction of the glycopeptide drug with biological membranes is greatly influenced by both the geometry and degree of glycosylation. Our previous experience with glycopeptide GPCR agonists of a similar size indicates that the degree of glycosylation (mono-vs disaccharide) will not greatly affect interaction with the MAS receptor or its activation.

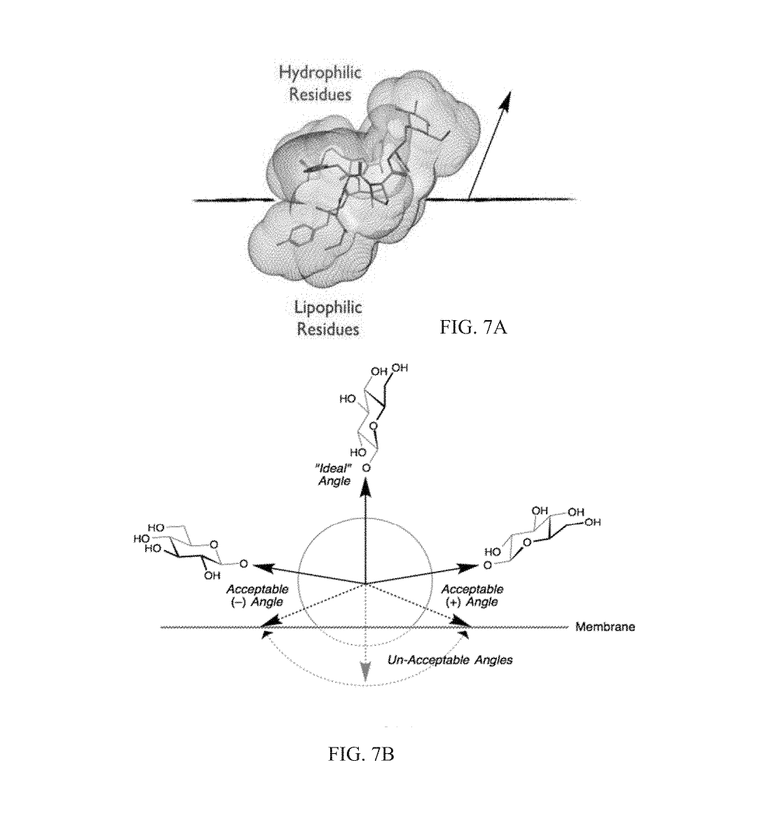

Membrane-bound conformations of the Ang(1-7)-based glycopeptides were modeled in silico by ¹H-NMR NOESY measurements in the presence of d₂₅-SDS micelles. Using derived H-H distance constraints, a highly amphipathic folded structure was characterized. As illustrated in FIG. 7A, a Solvent Accessible Surface Area was constructed using the MOE® software package with the AMBER-99 force field to illustrate the resulting amphipathicity of the U-shaped folded structure. The uncharged lipophilic residues Val-Tyr-Ile are at the bottom of the “U” and insert into the membrane while charged “ends” protrude into the aqueous compartment. The “amphipathic moment” is suggested by the arrow.

FIG. 7B illustrates the MOE® calculations indicating that the linkage geometries of the saccharide and peptide chain can modify interactions of the resulting amphipathic glycopeptide with biological membranes prior to “docking” with the Mas receptor. D- or L-Serine, D- or L-Threonine, and D- or L-allo-Threonine, as well as D- or L-Cysteine orient the glycoside at different angles relative to the surface of the membrane.

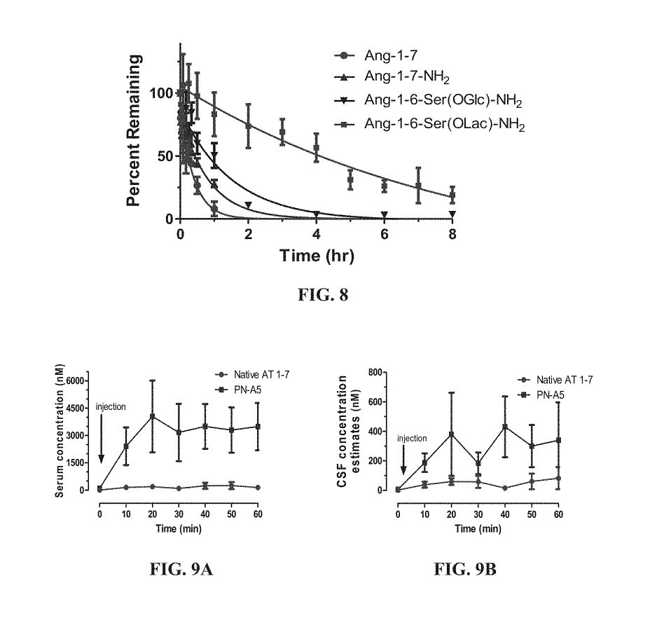

Based on these calculations, native Ang(1-7), Ang(1-7) having a C-terminal amino group (Ang 1-7-NH₂; SEQ ID NO: 3; “PN-A2”), PN-A5 (Ang 1-6-Ser(OGlc)-NH₂; SEQ ID NO: 13), and Ang 1-6-Ser(OLac)-NH₂ (Ang 1-6-Ser(OLac)-NH₂; SEQ ID NO: 13) were produced and the serum half-life tested. Serum half-life was assessed by incubating 100 μM of each peptide in mouse serum for eight hours. Aliquots were withdrawn at the indicated time intervals and the peptide concentration was determined using HPLC-MS and expressed as a percentage of the initial concentration. As illustrated in FIG. 8 and Table 2, glycosylation significantly improved the serum half-life of the Ang(1-7) derivatives.

TABLE 2
In Vitro Serum Half-Life Assay

| Peptide | Half-life | |
|---|---|---|
| Native Ang(1-7) | 14 | min |
| Ang 1-7-NH₂ (PN-A2) | 21 | min |
| Ang 1-6-Ser(OGlc)-NH₂ (PN-A5) | 1 | hour |
| Ang 1-6-Ser(OLac)-NH₂ (PN-A6) | 5.8 | hours |

Based on these findings, the in vivo serum stability and BBB penetration was assessed in vivo for Ang(1-7) and PN-A5. The peptides (10 mg/kg) or vehicle control were individually subcutaneously injected into naïve mice. Serum concentrations were determined every 10 minutes by HPLC-MS using a 20-30 μl blood sample. Ang(1-7) and PN-A5 were found to reach a maximum serum concentrations of about 200 nM and about 3,500 nM, respectively (FIG. 9A). CSF samples were simultaneously withdrawn from the same animals via a microdialysis probe and assayed for the peptide concentration and corrected for basal CSF levels. Ang(1-7) and PN-A5 were found to reach a maximum CSF concentrations of about 50 nM and about 400 nM, respectively (FIG. 9B).

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A method for treating a traumatic brain injury in a subject comprising administering a therapeutically effective amount of an oligopeptide having the formula: A¹-A²-A³-A⁴-A⁵-A⁶-A⁷-A⁸ (SEQ ID NO:1) wherein
A¹ is selected from the group consisting of aspartic acid, glutamic acid, alanine, and glycosylated forms thereof;
A² is selected from the group consisting of arginine, histidine, lysine, and glycosylated forms thereof;
A³ is selected from the group consisting of valine, alanine, isoleucine, leucine, and glycosylated forms thereof;
A⁴ is selected from the group consisting of tyrosine, phenylalanine, tryptophan, and glycosylated forms thereof;
A⁵ is selected from the group consisting of isoleucine, valine, alanine, leucine, and glycosylated forms thereof;
A⁶ is selected from the group consisting of histidine, arginine, lysine, and glycosylated forms thereof;
A⁷ is selected from the group consisting of proline, glycine, serine, and glycosylated forms thereof; and
A⁸ can be present or absent, wherein when A⁸ is present, A⁸ is selected from the group consisting of serine, threonine, hydroxyproline, and glycosylated forms thereof,

2. The method of claim 1, wherein the traumatic brain injury is a concussion.

3. The method of claim 1, wherein the traumatic brain injury is a penetrating brain injury.

4. The method of claim 1, wherein (a) A⁷ is terminated with an amino group and A⁸ is absent or (b) A⁸ is terminated with an amino group.

5. The method of claim 1, wherein at least one of A¹-A⁸ is glycosylated with a monosaccharide or disaccharide.

6. The method of claim 5, wherein at least one of the monosacharides or disaccharides is selected from the group consisting of glucose, galactose, xylose, fucose, rhamnose, lactose, cellobiose, and melibiose.

7. The method of claim 5, wherein (a) A⁷ is terminated with an amino group and A⁸ is absent or (b) A⁸ is terminated with an amino group.

8. The method of claim 1, wherein A⁸ is glycosylated with a monosaccharide or disaccharide or A⁸ is absent and A⁷ is glycosylated with a monosaccharide or disaccharide.

9. The method of claim 8, wherein at least one of the monosacharides or disaccharides is selected from the group consisting of glucose, galactose, xylose, fucose, rhamnose, lactose, cellobiose, and melibiose.

10. The method of claim 8, wherein (a) A⁷ is terminated with an amino group and A⁸ is absent or (b) A⁸ is terminated with an amino group.

11. The method of claim 1, wherein (a) A⁷ is a serine or a glycosylated form thereof and A⁸ is absent or (b) A⁸ is serine or a glycosylated form thereof.

12. The method of claim 11, wherein (a) A⁷ is glycosylated with glucose or lactose and A⁸ is absent or (b) A⁸ is glycosylated with glucose or lactose.

13. The method of claim 11, wherein (a) A⁷ is terminated with an amino group and A⁸ is absent or (b) A⁸ is terminated with an amino group.

14. The method of claim 1, wherein the oligopeptide is selected from the group consisting of PN-A2, PN-A3, PN-A4, PN-A5, and PN-A6.

15. The method of claim 14, wherein the oligopeptide is PN-A5.

16. The method of claim 14, wherein the oligopeptide is PN-A6.

17. The method of claim 1, wherein the oligopeptide comprises at least one D-amino acid.

18. The method of claim 1, wherein each amino acid is a D-amino acid.


SEQUENCE LISTING (27 sequences)

SEQ ID NO: 1 - protein, 8 aa, Artificial Sequence
  Description of Artificial Sequence Synthetic peptide
  MOD_RES (1)..(1): Aspartic acid, glutamic acid, alanine or a glycosylated form thereof
  MOD_RES (2)..(2): Arginine, histidine, lysine or a glycosylated form thereof
  MOD_RES (3)..(3): Valine, alanine, isoleucine, leucine or a glycosylated form thereof
  MOD_RES (4)..(4): Tyrosine, phenylalanine, tryptophan or a glycosylated form thereof
  MOD_RES (5)..(5): Isoleucine, valine, alanine, leucine or a glycosylated form thereof
  MOD_RES (6)..(6): Histidine, arginine, lysine or a glycosylated form thereof
  MOD_RES (7)..(7): Proline, glycine, serine or a glycosylated form thereof
  MOD_RES (8)..(8): Can be absent or serine, threonine, hydroxyproline or a glycosylated form thereof
  See specification as filed for detailed description of substitutions and preferred embodiments
XXXXXXXX

SEQ ID NO: 2 - protein, 7 aa, Homo sapiens
DRVYIHP

SEQ ID NO: 3 - protein, 7 aa, Artificial Sequence
  Description of Artificial Sequence Synthetic peptide
  MISC_FEATURE (7)..(7): Glycosylated
DRVYIHP

SEQ ID NO: 4 - protein, 7 aa, Artificial Sequence
  Description of Artificial Sequence Synthetic peptide
  MISC_FEATURE (7)..(7): Carboxy terminal NH2
DRVYIHP

SEQ ID NO: 5 - protein, 7 aa, Artificial Sequence
  Description of Artificial Sequence Synthetic peptide
  MISC_FEATURE (7)..(7): Glycosylated
  MISC_FEATURE (7)..(7): Carboxy terminal NH2
DRVYIHP

SEQ ID NO: 6 - protein, 8 aa, Artificial Sequence
  Description of Artificial Sequence Synthetic peptide
DRVYIHPS

SEQ ID NO: 7 - protein, 8 aa, Artificial Sequence
  Description of Artificial Sequence Synthetic peptide
  MISC_FEATURE (8)..(8): Glycosylated
DRVYIHPS

SEQ ID NO: 8 - protein, 8 aa, Artificial Sequence
  Description of Artificial Sequence Synthetic peptide
  MISC_FEATURE (8)..(8): Carboxy terminal NH2
DRVYIHPS

SEQ ID NO: 9 - protein, 8 aa, Artificial Sequence
  Description of Artificial Sequence Synthetic peptide
  MISC_FEATURE (8)..(8): Glycosylated
  MISC_FEATURE (8)..(8): Carboxy terminal NH2
  See specification as filed for detailed description of substitutions and preferred embodiments
DRVYIHPS

SEQ ID NO: 10 - protein, 7 aa, Artificial Sequence
  Description of Artificial Sequence Synthetic peptide
DRVYIHS

SEQ ID NO: 11 - protein, 7 aa, Artificial Sequence
  Description of Artificial Sequence Synthetic peptide
  MISC_FEATURE (7)..(7): Glycosylated
DRVYIHS

SEQ ID NO: 12 - protein, 7 aa, Artificial Sequence
  Description of Artificial Sequence Synthetic peptide
  MISC_FEATURE (7)..(7): Carboxy terminal NH2
DRVYIHS

SEQ ID NO: 13 - protein, 7 aa, Artificial Sequence
  Description of Artificial Sequence Synthetic peptide
  MISC_FEATURE (7)..(7): Glycosylated
  MISC_FEATURE (7)..(7): Carboxy terminal NH2
  See specification as filed for detailed description of substitutions and preferred embodiments
DRVYIHS

SEQ ID NO: 14 - protein, 7 aa, Artificial Sequence
  Description of Artificial Sequence Synthetic peptide
ARVYIHP

SEQ ID NO: 15 - protein, 7 aa, Artificial Sequence
  Description of Artificial Sequence Synthetic peptide
  MISC_FEATURE (7)..(7): Glycosylated
ARVYIHP

SEQ ID NO: 16 - protein, 7 aa, Artificial Sequence
  Description of Artificial Sequence Synthetic peptide
  MISC_FEATURE (7)..(7): Carboxy terminal NH2
ARVYIHP

SEQ ID NO: 17 - protein, 7 aa, Artificial Sequence
  Description of Artificial Sequence Synthetic peptide
  MISC_FEATURE (7)..(7): Glycosylated
  MISC_FEATURE (7)..(7): Carboxy terminal NH2
ARVYIHP

SEQ ID NO: 18 - protein, 8 aa, Artificial Sequence
  Description of Artificial Sequence Synthetic peptide
ARVYIHPS

SEQ ID NO: 19 - protein, 8 aa, Artificial Sequence
  Description of Artificial Sequence Synthetic peptide
  MISC_FEATURE (8)..(8): Glycosylated
ARVYIHPS

SEQ ID NO: 20 - protein, 8 aa, Artificial Sequence
  Description of Artificial Sequence Synthetic peptide
  MISC_FEATURE (8)..(8): Carboxy terminal NH2
ARVYIHPS

SEQ ID NO: 21 - protein, 8 aa, Artificial Sequence
  Description of Artificial Sequence Synthetic peptide
  MISC_FEATURE (8)..(8): Glycosylated
  MISC_FEATURE (8)..(8): Carboxy terminal NH2
ARVYIHPS

SEQ ID NO: 22 - protein, 7 aa, Artificial Sequence
  Description of Artificial Sequence Synthetic peptide
ARVYIHS

SEQ ID NO: 23 - protein, 7 aa, Artificial Sequence
  Description of Artificial Sequence Synthetic peptide
  MISC_FEATURE (7)..(7): Glycosylated
ARVYIHS

SEQ ID NO: 24 - protein, 7 aa, Artificial Sequence
  Description of Artificial Sequence Synthetic peptide
  MISC_FEATURE (7)..(7): Carboxy terminal NH2
ARVYIHS

SEQ ID NO: 25 - protein, 7 aa, Artificial Sequence
  Description of Artificial Sequence Synthetic peptide
  MISC_FEATURE (7)..(7): Glycosylated
  MISC_FEATURE (7)..(7): Carboxy terminal NH2
ARVYIHS

SEQ ID NO: 26 - protein, 7 aa, Artificial Sequence
  Description of Artificial Sequence Synthetic peptide
  MOD_RES (1)..(1): Asp, Glu, Ala, Asn, Pro or Gly
  MISC_FEATURE (1)..(1): May be unmodified or may be glycosylated
  MOD_RES (2)..(2): Arg, Lys or Ala
  MISC_FEATURE (2)..(2): May be unmodified or may be glycosylated
  MOD_RES (3)..(3): Nle, Val, Leu, Ile, Ala, Gly, Lys, Pro or Tyr
  MISC_FEATURE (3)..(3): May be unmodified or may be glycosylated
  MOD_RES (4)..(4): Tyr, Ser, Thr or Ala
  MISC_FEATURE (4)..(4): May be unmodified or may be glycosylated
  MOD_RES (5)..(5): Ile, Val, Leu, Nle, Ala or Gly
  MOD_RES (6)..(6): His or Arg
  MISC_FEATURE (6)..(6): May be unmodified or may be glycosylated
  MOD_RES (7)..(7): Pro, Ser or Cys
  MISC_FEATURE (7)..(7): May be unmodified, may be glycosylated, may have a carboxy terminal NH2 or may be both glycosylated and have a carboxy terminal NH2
  See specification as filed for detailed description of substitutions and preferred embodiments
XXXXXXX

SEQ ID NO: 27 - protein, 8 aa, Artificial Sequence
  Description of Artificial Sequence Synthetic peptide
  MOD_RES (1)..(1): Asp, Glu, Ala, Asn, Pro or Gly
  MISC_FEATURE (1)..(1): May be unmodified or may be glycosylated
  MOD_RES (2)..(2): Arg, Lys or Ala
  MISC_FEATURE (2)..(2): May be unmodified or may be glycosylated
  MOD_RES (3)..(3): Nle, Val, Leu, Ile, Ala, Gly, Lys, Pro or Tyr
  MISC_FEATURE (3)..(3): May be unmodified or may be glycosylated
  MOD_RES (4)..(4): Tyr, Ser, Thr or Ala
  MISC_FEATURE (4)..(4): May be unmodified or may be glycosylated
  MOD_RES (5)..(5): Ile, Val, Leu, Nle, Ala or Gly
  MOD_RES (6)..(6): His or Arg
  MISC_FEATURE (6)..(6): May be unmodified or may be glycosylated
  MOD_RES (7)..(7): Pro or Ala
  MOD_RES (8)..(8): Phe, Ser, Cys, Ile or Tyr
  MISC_FEATURE (8)..(8): May be unmodified, may be glycosylated, may have a carboxy terminal NH2 or may be both glycosylated and have a carboxy terminal NH2
  See specification as filed for detailed description of substitutions and preferred embodiments
XXXXXXXX